(19) United States
(12) Patent Application Publication
(10) Pub. No.: US 2006/0173367 A1
(43) Pub. Date: Aug. 3, 2006

(54) Equine fitness monitoring

(75) Inventors: Andrew K. Stuart, Brentwood (AU); David Evans, Kiama (AU)

Correspondence Address: EDWARDS & ANGELL, LLP, P.O. BOX 55874, BOSTON, MA, 02205, US

(73) Assignee: Equitronic Technologies PTY LTD, Bicton (AU)

(21) Appl. No.: 10/550,854

(22) PCT Filed: Mar. 26, 2004
(86) PCT No.: PCT/AU04/00380

§ 371 Date: Sep. 22, 2005

(30) Foreign Application Priority Data
Dec. 17, 2003 (AU) 2003906956
Mar. 27, 2003 (AU) 2003901421

(51) Int. Cl. A61B 5/02

(52) U.S. Cl. 600/508

(57) ABSTRACT

Apparatus for determining the health or fitness, under an exercise load, of an animal such a horse comprises: a first sensor (incorporated in module (3) and having electrodes (5, 6)), positioned in blanket (1) under saddle (2a) of a horse, generating physiological data, e.g. breathing or heart rate, blood pressure or flow, temperature, etc; a second sensor, also incorporated in module (3), for generating position data, e.g. a GPS sensor having antenna (11). By using an algorithm, a fitness indicator such as velocity at a heart rate of 200 beats per minute (V-200) can then be derived using data from the sensors. Lameness, disease or poor physiological potential of the animal can thus be identified.

### BACKGROUND OF THE INVENTION

The present invention relates to apparatus and a method for monitoring the status of a horse, and in particular to apparatus including a blanket incorporating a sensor, such as a heart rate sensor.

### DESCRIPTION OF THE PRIOR ART

The reference to any prior art in this specification is not, and should not be taken as, an acknowledgment or any form of suggestion that the prior art forms part of the common general knowledge.

Monitoring equine fitness is extremely important in ensuring animal health and to provide performance management. For example, it has been shown that there is value in monitoring certain parameters over time to provide a more quantitative assessment of health. These parameters can be measured via heart rate monitors and the like to provide maximum effectiveness.

Typically, monitoring is achieved by passively monitoring the animal during training, based on feedback from trainers and jockeys via trackwork results, or via controlled studies.

In the case of controlled studies, this may achieved using treadmill studies, which rely on the implementation of a standardised protocol in controlled environments. In particular, such tests are typically performed based on precise exercise protocols, with the speeds and durations for each step of an exercise test being highly repeatable.

From such studies, it has been suggested that the velocity at a heart rate of 200 beats per minute (hereinafter referred to as “V200”) can be used as an expression of the maximal aerobic power of the horse. As an expression for the heart rate/velocity relationship, the interpolated or extrapolated velocity at heart rate 200 (V200) may be used as it is close to the workload at which onset of blood lactic acid accumulation (anaerobic threshold) occurs. Such tests have proved to be useful and reliable tool for evaluation of training effects, and have shown a negative correlation between V200 and treadmill running speed, suggesting that faster horses attain a heart rate of 200 beats per minutes at a lower speed than slower horses.

However, such test have also shown a number of problems. For example, the horses need to be acclimatised to treadmill exercise, and responses to acclimatisation are unpredictable in individual horses. In addition to this, locomotion during treadmill exercise is also different to that on the track. Stride frequencies at identical trot and gallop speeds are greater on a racetrack than on a treadmill and such studies, do not therefore typically reflect practical exercise conditions.

An example of a field test is described in Kobayashi (1999), in which V200 was calculated with an incremental field exercise test in racehorses. This was the study reported the practical application of V200 for the evaluation of training effects in the young Thoroughbreds, but was limited to specific tests in limited environments, and did not therefore monitor the horse under standard conditions, which tends to lead to unreliability of results.

For example, environmental conditions, such as high and low ambient temperature and relative air humidity can be an important factor during the conduct of field exercise tests. Furthermore, field studies usually do not take into account air resistance that the racehorse has to face on the track, and provide only a limited range of measurements.

Such studies do not therefore represent practical conditions in which horses train or race. Thus, in Kobayashi, high V200 was found, due to a number of factors including:
high heart rates during trotting, which is indicative of excitability;
gait changes that were not carried out “smoothly”; and,
phases of rapid acceleration during gallop exercise.

Thus, whilst specific tests may be useful in examining a specific fitness area, under specific conditions, these tests are generally time consuming and of limited value in practical applications.

In addition to this, such tests generally require specialist equipment and does not therefore allow practical fitness testing of horses. For example, U.S. Pat. No. 6,504,483 which describes a system for electronically monitoring vital signs of a moving horse. This relies on the provision of monitoring equipment around a pre-designated track, thereby severely limiting the circumstances in which the device may be used.

Solutions have been proposed for allowing heart rate to be measured in situ. For example, U.S. Pat. No. 4,540,001 and U.S. Pat. No. 4,478,225 relate to the provision of a heart monitor for horses, with the heart monitor being incorporated either in the saddle itself or in a saddle girth. However, these techniques suffer from a number of drawbacks.

Firstly, saddles tend to be expensive, and providing such monitoring equipment therein further increases article cost. In addition to this, if a fault develops with the monitoring equipment, it can be costly to replace the equipment and the saddle.

Secondly, saddles tend to be subject to high stresses in use, thereby reducing the effectiveness of the monitoring equipment.

Thirdly, saddles in generally are not suitable for mounting monitoring equipment, and this tends to reduce the comfort of the saddles to both the rider and the horse, thereby impairing performance during testing.

This also tends to restrict the re-use of the apparatus with different horses, such that trainers will typically need a respective saddle for each horse.

### SUMMARY OF THE PRESENT INVENTION

In a first broad form the present invention provides apparatus for monitoring the status of a horse, wherein the apparatus includes:
(a) a blanket having a first sensor, the first sensor being adapted to generate indicating data indicative of at least one health status indicator; and,
(b) a second sensor for generating position data indicative of the position of the horse, wherein, in use, a processing system is adapted to determine the health status of the horse in response to the indicating data and the position data.

Typically the health status indicator includes at least one of the horse's:
(a) heart rate;
(b) blood pressure;
(c) temperature; breathing rate;
(d) blood flow rate; and,
(e) blood oxygenation levels.

Typically the second sensor is formed from a GPS sensor.

The second sensor is usually adapted to be worn by a rider in use, and wherein the blanket further includes a connector for coupling the second sensor to the blanket in use.

The second sensor may be provided in the blanket.

The blanket may further include a power supply for coupling to the first and second sensors.

The power supply typically includes at least one battery connected to a first part of an inductive coupling, and wherein, in use, the battery is recharged by connecting the first part of the inductive coupling to a second part of the inductive coupling, the second part being coupled to a power supply.

The blanket typically further includes a communications device coupled to the first and second sensors to thereby transfer at least one of the indicating and position data to a remote computer system.

The blanket usually further includes a store coupled to the first and second sensors to thereby store at least one of the indicating and position data to a remote computer system.

The apparatus can include a processing system coupled to at least one of the first and second sensors for at least partially analysing at least one of the indicating and the position data.

The processing system may be coupled to a display, the display being adapted to provide an indication to the rider in accordance with at least one of the indicating and the position data.

The first sensor can be a heart rate sensor and wherein the blanket includes at least one electrode coupled to the heart rate sensor and positioned so as to be in contact with the horse in use.

The blanket can include at least one wire embedded in the blanket material, the wire being adapted to connect the heart rate sensor to the at least one electrode.

The blanket may be a woven blanket and wherein the wire is integrated within the weave of the blanket.

The first sensor can be removably mounted to a pouch, the pouch including one or more connectors adapted to cooperate with corresponding detectors provided on the sensor, to thereby couple the sensor to the blanket.

In a second broad form the present invention provides apparatus for monitoring the status of a horse, wherein the apparatus includes a processing system adapted to:
(a) receive, from a first sensor provided in a horse blanket, indicating data indicative of at least one health status indicator;
(b) receive, from a second sensor, position data indicative of the position of the horse; and,
(c) determine the health status of the horse in accordance with the indicating data and the position data.

The processing system can be adapted to receive the position and indicating data from apparatus according to the first broad form of the invention.

The processing system can include a communications device for receiving the indicating and position data.

The processing system may determine the health status of the horse using a predetermined algorithm, the predetermined algorithm defining a relationship between the at least one health status indicator and movement of the horse.

The predetermined algorithm typically includes:
(a) determining at least a low heart rate during low speed exercise;
(b) determining a number of heart rates during high speed exercise;
(c) perform linear regression to calculate a linear regression line:
(d) calculate, using the linear regression line, the velocities at at least one of:
   (i) heart rates of 200 beats per minute (V200); and,
   (ii) HRmax (VHRmax); and,
(e) determine a fitness indicator in accordance with the calculated at least one velocity.

The line regression line can determined in accordance with:
HR=a+bV,
where HR=heart rate;

a=constant;

b=constant; and,

V=velocity.

The method usually further includes deleting any outlier values, which can include at least one of:
(a) deleting all results with a velocity of less than 40 kph;
(b) deleting all results during the period after exercise (from the time of occurrence of HRmax);
(c) deleting all data equal to at least one of:
   (i) HRmax;
   (ii) HRmax−1;
   (iii) HRmax−2; and,
   (iv) HRmax−3;
(d) deleting all data where there has been an increase in velocity, but that increase was not accompanied by an increase in HR;
(e) deleting any data points which have a HR that is more than 10 beats per minute above the regression line at that speed, and recalculate the regression line if such outliers are deleted.

The processing system can be adapted to obtain indicating data and position data relating to a number of horses, the processing system being adapted to determine the health status of each of the number of horses.

In a third broad form the present invention provides apparatus for monitoring the status of a horse, wherein the apparatus includes a processing system adapted to:
(a) receive, from a first sensor, indicating data indicative of the heart rate of the horse;
(b) receive, from a second sensor, position data indicative of the position of the horse;
(c) determine from the position data, movement data indicative of the rate of movement of the horse; and,
(d) determine the health status of the horse in accordance with a predetermined algorithm, the predetermined algorithm defining a relationship between the heart rate and the rate of movement of the horse.

The predetermined algorithm typically includes:
(a) determining at least a low heart rate during low speed exercise;
(b) determining a number of heart rates during high speed exercise;
(c) perform linear regression to calculate a linear regression line:
(d) calculate, using the linear regression line, the velocities at at least one of:
   (i) heart rates of 200 beats per minute (V200); and,
   (ii) HRmax (VHRmax); and,
(e) determine a fitness indicator in accordance with the calculated at least one velocity.

In a fourth broad form the present invention provides a system for monitoring the status of a horse, wherein the system includes:
(a) a blanket having a first sensor, the first sensor being adapted to generate indicating data indicative of at least one health status indicator; and,
(b) a second sensor for generating position data indicative of the position of the horse; and,
(c) a processing system, the processing system being responsive to the indicating and position data to thereby determine the health status of the horse.

The system may including apparatus according to the first broad form of the invention.

In a fifth broad form the present invention provides a method of monitoring the health status of a horse, wherein the method includes:
(a) using a blanket having a first sensor to generate indicating data indicative of at least one health status indicator; and,
(b) using a second sensor to generate position data indicative of the position of the horse; and,
(c) determining the health status of the horse in response to the indicating data and the position data.

The method can be performed using the apparatus of the first broad form of the invention.

In a sixth broad form of the invention provides a method of monitoring the health status of a horse, wherein the method includes, in a horse blanket:
(a) generating indicating data using a first sensor, the indicating data being indicative of at least one health status indicator;
(b) obtaining position data from a second sensor, the position data being indicative of the position of the horse; and,
(c) providing the indicating data and the position data to a processing system, the processing system being responsive to the indicating data and the position data to determine the health status of the horse.

The method can be performed using the apparatus of the first broad form of the invention.

In a seventh broad form the present invention provides a method of monitoring the health status of a horse, wherein the method includes, in a processing system:
(a) receiving, from a first sensor provided in a horse blanket, indicating data indicative of at least one health status indicator;
(b) receiving, from a second sensor, position data indicative of the position of the horse; and,
(c) determining the health status of the horse in accordance with the indicating data and the position data.

The method can be performed using the apparatus of the first broad form of the invention.

In an eighth broad form the present invention provides apparatus for monitoring the status of a horse, wherein the apparatus includes a processing system adapted to:
(a) receive, from a first sensor, indicating data indicative of the heart rate of the horse;
(b) receive, from a second sensor, position data indicative of the position of the horse;
(c) determine from the position data, movement data indicative of the rate of movement of the horse; and,
(d) determine the health status of the horse in accordance with a predetermined algorithm, the predetermined algorithm defining a relationship between the heart rate and the rate of movement of the horse.

The predetermined algorithm typically includes:
(a) determining at least a low heart rate during low speed exercise;
(b) determining a number of heart rates during high speed exercise;
(c) perform linear regression to calculate a linear regression line:
(d) calculate, using the linear regression line, the velocities at at least one of:
   (i) heart rates of 200 beats per minute (V200); and,
   (ii) HRmax (VHRmax); and,
(e) determine a fitness indicator in accordance with the calculated at least one velocity.

The method can be performed using the apparatus of the first broad form of the invention.

### BRIEF DESCRIPTION OF THE DRAWINGS

An example of the present invention will now be described with reference to the accompanying drawings, in which:

FIG. 1 is a schematic diagram of a first example of a saddle blanket system incorporating apparatus for monitoring horses;

FIG. 2 is a schematic side view of the blanket system of FIG. 1;

FIG. 3 is a schematic diagram of an example of apparatus for analysing signals generated by the blanket system of FIG. 1;

FIG. 4 is a schematic diagram of a second example of a saddle blanket system incorporating apparatus for monitoring horses;

FIG. 5 is a schematic side view of the blanket system of FIG. 4;

FIG. 6 is a schematic diagram of a charging system;

FIG. 7 is a schematic plan view of the blanket system of FIG. 4;

FIG. 8 is a schematic diagram of an example of a distributed architecture for monitoring a number of horses;

FIG. 9 is a schematic diagram of an example of a processing system of FIG. 8;

FIG. 10 is an example of heart rate and velocity determined using the system of FIG. 1 or FIG. 4;

FIGS. 11A-11D are graphs showing a determined linear regression for the data shown in FIG. 10;

FIG. 12 is a graph showing data collected from trials of the system of FIG. 1.

### DETAILED DESCRIPTION OF THE PREFERRED EMBODIMENTS

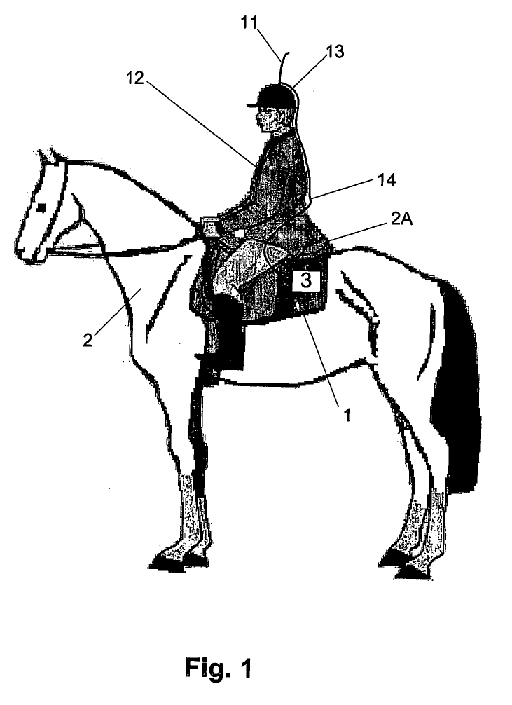

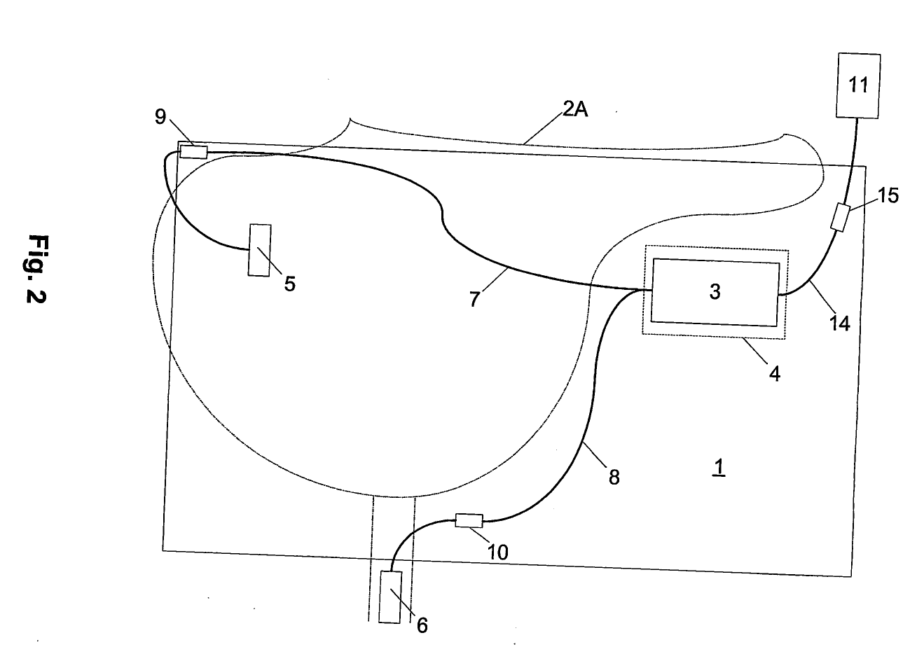

An example of a saddle blanket incorporating apparatus for monitoring horse vital signs and/or position will now be described with reference to FIGS. 1 and 2.

As shown, the blanket 1 is designed to be worn by a horse 2, under a saddle 2A. The blanket 1 incorporates a module 3, which is sewn into the blanket, as shown at 4. The blanket generally has a layer of foam, allowing the module 3 to be recessed therein, such that the blanket lies flush against the horse, without the module protruding inwards, and digging into the horse. The blanket is also typically formed from specialised materials to enhance function and durability, for example to provide shock absorption, and air flow.

The module 3 is connected to two electrodes 5, 6, via respective cables 7, 8. In order to ensure correct sensing of the heart rate the electrodes 5, 6 are positioned respectively on the top shoulder of the horse and one placed above the heart, just below the girth. In this example, the electrodes may be provided separately to the blanket, with respective connectors 9, 10 being provided, to allow the electrodes 5, 6 to be fixed to the horse, and then connected to the module 3, when the blanket is placed on the horse 2. Normal industry practise is to place a folded towel underneath the saddle blanket to absorb the sweat. In this instance, Velcro provided on the towel, in the electrode position, can be used to hold the electrode in the correct position. Alternatively, however, Velcro could be placed on the underside of the blanket to hold the electrode in place. Similarly, for the girth position, the electrode can be built into a girth cover which would be slid over the girth as the saddle is placed on the horse. In both cases, the cables extend through the blanket to the relevant electrode position, thereby connecting the electrode to the module 3.

In used, the horse's skin can simply be wetted, before the electrodes are positioned thereon. The electrodes are then held in place, as described above, thereby providing a simple means for ensuring correct electrode positioning.

In this example, the module 3 is also connected to an antenna 11, which is used to receive signals indicative of the horse's position, such as GPS signals. The antenna 11 is mounted to the jockey 12, for example on the helmet 13, and is connected to the module 3 via a cable 14. The antenna is situated on the helmet to ensure successful receiving of the GPS signals, in particular as the horse's bulk will shield antennas positioned in the blanket to some extent, although alternative positions may be used. The cable 14 includes a connector 15, which allows the jockey to mount the horse before connecting the GPS system to the blanket. In addition to this, the cable 14 and connector 15 also act as a safety feature, such that if a rider falls off the horse, the cable 14 will pull out in a straight direction, disengaging the connector, so that there is a reduced chance of jerk to rider.

In use, the module 3 typically includes processing electronics which is adapted to monitor signals generated by the electrodes 5, 6 to determine the heart rate therefrom, as well as to monitor position indication signals received from the GPS system 11.

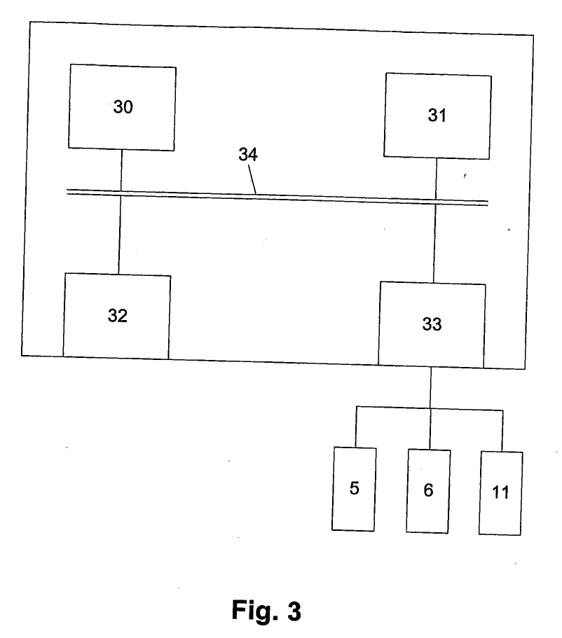

Persons skilled in the art will appreciate that a number of different configurations may be used to obtain this functionality. In one example, shown in FIG. 3, the processing electronics include a processor 30, a memory 31, a communications system 32, and an external interface 33, coupled together via a bus 34. In use, signals from the electrodes 5, 6, and the antenna 11 are received via the external interface 33, and passed on to the processor 30, for preliminary processing.

In particular, in this example, the processor 30 is adapted to determine the current heart rate and position of the horse, and then store these as heart rate and position data in the memory 31. It will therefore be appreciated that the processing electronics can be formed from custom hardware, such as a DSP, and corresponding memory, and/or through the use of applications software operating on a suitable generic processing system.

The heart rate and position data can then be further processed to allow a health status of the horse to be determined. This is typically performed by a remote processing system, such as a computer or the like. Accordingly, the communications system 32 can be used to transfer the data to the computer system. This can be performed via a wireless radio based transmission system. This may include short range communications systems, such as Wi-Hi, Bluetooth, or the like. Alternatively, long range radio connections, such as the GSM or other mobile phone networks, can be used. As a further alternative wired connections can be used to transfer data at the end of an exercise period, as will be appreciated by persons skilled in the art.

In order to track horses that are in training over a long distance, for example over a typical training track, which can be up to 10 Km in length, long range radio communications is typically used. This can be achieved using a high powered radio modem, which will typically utilise an antenna sewn into the saddle blanket. The antenna typically runs down the back of the saddle blanket, but may be formed from shorter stubby antennas attached to one of the modules, such as the module 3.

The polarisation of the antenna will depend on the size and shape of the horse, and different configurations may therefore be provided in different sizes of blankets. A circularly polarised antenna provided at the receiving end, helps overcome any problems that may arise due to polarisation of the transmitted signal.

Because the orientation of the horse can be in any direction, the antenna in the saddle blanket is preferably provided on both sides of the horse. The antenna may also be shielded to help reduce exposure of the horse to RF signals, as well as to help project signals.

It will also be appreciated that the processing electronics provided in the module 3 may perform the analysis, as will be described in more detail below.

In any event, the position data is used to determine the rate of movement of the horse over a time period, with this information being used in conjunction with the corresponding heart rate, to determine the horse health status.

This can be achieved without the use of a standardised exercise test protocol, and associated analysis procedure are used in accordance with the following methodology:
1. Identify the lowest pair of heart rates during trotting;
2. Identify the maximal heart rate (highest) (HRmax);
3. Delete all results with velocity less than 40 kph;
4. Delete all results during the period after exercise (from the time of occurrence of HRmax);
5. Delete all data equal to HRmax, or equal to HRmax−1, HRmax−2, or HRmax−3;
6. Delete all data where there has been an increase in velocity, but that increase was not accompanied by an increase in HR;
7. With the trotting and remaining data from high speed exercise, calculate the linear regression line;
   HR=a+b (velocity)
8. Inspect the scatter plot and linear regression line. Delete any data points which have a HR that is more than 10 beats per minute above the regression line at that speed (i.e., delete residuals of more than +10), and recalculate the regression line if such outliers are deleted.
9. Using the linear regression equation, calculate the velocities at heart rates of 200 beats per minute (V200), and at the HRmax (VHRmax):

The trotting heart rate is determined after the horse has been trotting for at least three minutes, as this allows the spleen reaction to die down after the horse has commenced exercise.

In addition to this, when the horse reaches the highest heart rate, this will typically result in the detection of a group of five heart rates, accompanied by increasing velocities, in which case the first heart rate is used.

The algorithm is therefore typically implemented using a processing system to assist with the data analysis, which may therefore be performed automatically, manually, or through a combination of automated or manual operation.

Accordingly, in use, the blanket and associated sensors can be mounted to the horse substantially as described above, allowing information on the horse health status to be determined whilst the horse is being ridden. The collected data can then be transferred to a computer system, either whilst the horse is being ridden, or at the end of the ride, thereby allowing the health status to be immediately determined.

Variations

A number of alternatives and additional features will now be described with respect to the accompanying drawings.

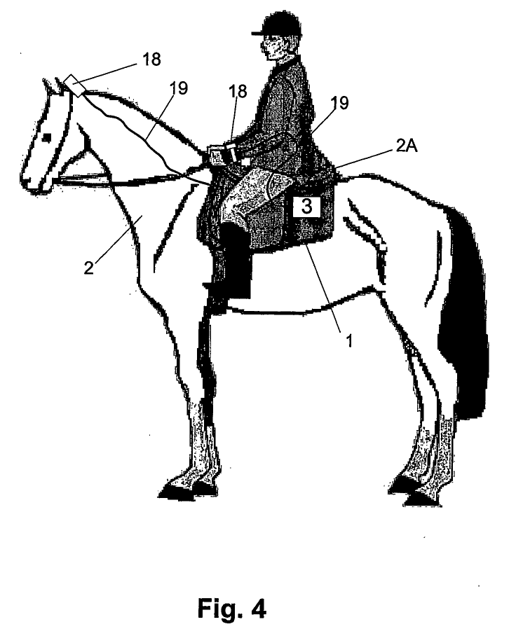

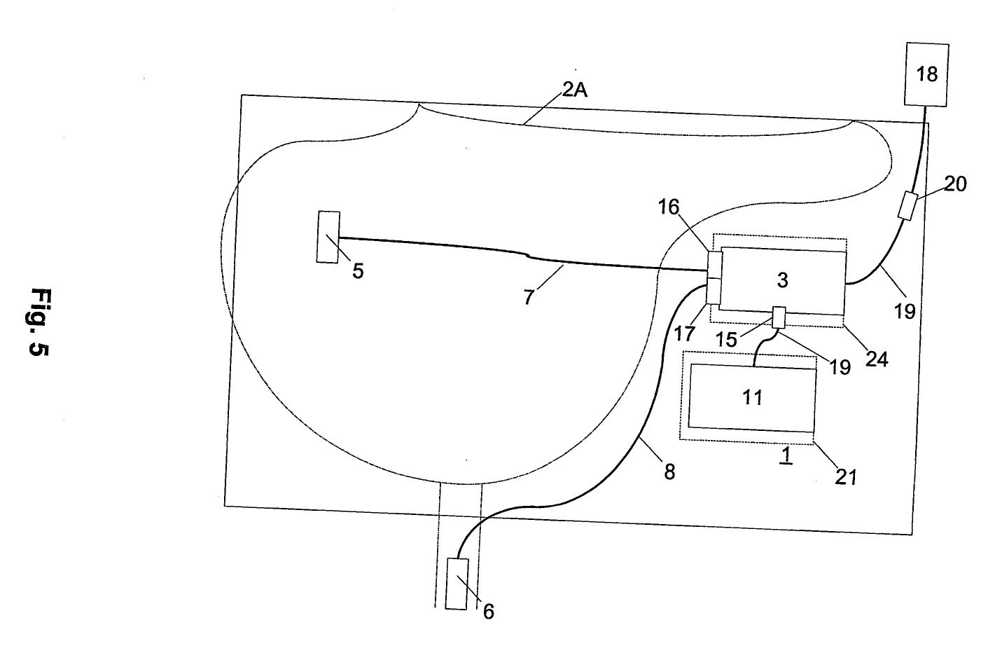

In a second example of the saddle blanket, shown in FIG. 4 and 5, the module 3 is positioned in a pouch 24, thereby allowing the module to be removed from the blanket. In order to achieve this, the module is connected to the cables 7, 8, and 14 via respective connectors 15, 16, 17, as shown.

This allows the module to be removed and replaced or repaired, if required. In addition to this, the module can be removed to allow the heart rate and position data to be downloaded therefrom. In this example, the communications system 32 may therefore correspond to a connection, such as a USB port, or the like. Alternatively, the memory 31 can be provided in the form or a removable media, such as a smart card, or the like, from which the data can be downloaded on to a computer.

In this example, the electrode 5 is integrally formed within the blanket, with the electrode 6 remaining attached to the blanket at all times, thereby removing the requirement for the connectors 9, 10.

Furthermore, the system includes one or more display devices 18 for providing status information to the rider. In this example, the display devices can be connected to the processing electronics, either wirelessly, for example by using a short-range communications protocol, such as Bluetooth, or through the use of cables 19, as shown. In this case, if one of the display devices is worn by the rider as shown, it is typical for the cable 19 to be provided with a connector 20, adapted to disengage if the rider falls.

The displays may be provided either behind the ears of the horse, or on the rider, as shown, and may be implemented using liquid crystal displays, a heads up display (HUD), or the like. Preferably the display is readable in the dark and in daylight conditions, in which case a backlit display may be needed, which will place extra demands on the battery life. Accordingly, the display is typically provided with a push button backlight activation system, which will activate the backlight for a predetermined time period, allowing the display to be viewed regardless of ambient conditions.

The display is light enough to mount on the horses bridle on the back of the horses head, or may be worn by the rider, as shown. If mounted on the horse, this is typically achieved using some form of mounting strap so as to not upset the horse.

The information displayed to the rider will depend on the respective implementation, and the information required. For example, the information may include no more that the current heart rate and the current position or speed of the horse.

Alternatively, the display devices can be adapted to show the current health status of the horse. This can be achieved by having the health status, as determined real time by the remote computer, relayed back to the module 3, via the communications system 32, and then displayed as required. Alternatively, the health status can be determined by having the processing electronics perform analysis of the heart rate and position data, substantially as described above, to thereby determine the health status of the horses and display this to the user. In this case, the health status may be downloaded to a remote computer system at the end of the ride, in the same manner as the heart rate and position data, as described above.

In addition to this, the displays can incorporate a communications device, to thereby allow communication between the rider and a trainer, owner or the like. This may be achieved using voice and text messages, or the like. Pre-programmed work routines and instructions could also be display to the rider.

As a further variation, positional information determined from the GPS system can also be used to initiate messages, functions and the like. This can be used for example, to prompt the rider to perform predetermined actions, such as trotting, cantering, or the like.

The GPS antenna 11 may also be integrated into the horse blanket, or form part of an integrated GPS system, including a dedicated GPS processor and an antenna which is provided within a respective pouch 21, as shown. Alternatively, a dedicated GPS processor can be provided on or in the rider's helmet with an associated transmitter and power supply, to allow the position data to be transferred to the module 3, for example, using a Bluetooth communications link.

It will be appreciated that other parts of the module 3 and the associated processing electronics may be provided on or in the rider's helmet.

In this case, a wireless connection may be used to link the GPS system to the display 18 and/or the module 3. In this case, the GPS system may be provided with a unique identifier, to allow module 3 to ensure signals are received from the correct GPS system. In this case, by associating the identifier with the jockey, this allows the system to distinguish the jockey, and associate this with an identifier provided for the horse in the display device 18 or the electronics module 3, as described below.

Typically, whilst there would be no more than 30-100 horses training at a time, an electronic identifier provided with the GPS system provides a significant number of combinations allowing horses, blanket systems and jockeys to be identified uniquely during the training session.

In one example, differential GPS is used to provide greater accuracy in determination of horse position. However, this level of accuracy is not generally required and standard accuracy GPS may be used, removing the need for the antenna 11.

In general, GPS systems require recent satellite information to be able to provide accurate information. This initiation process typically takes about three to four minutes. Accordingly, in one example, the remote processing system 50 can be used to provide the latest information to the onboard GPS system via a communications link, thereby reducing start up time. This may typically be performed while the blanket is being stored, at which time it is also typical to perform a systems check to verify operational status. This may be carried out whilst the blankets are on a special rack for charging, as described in more detail below.

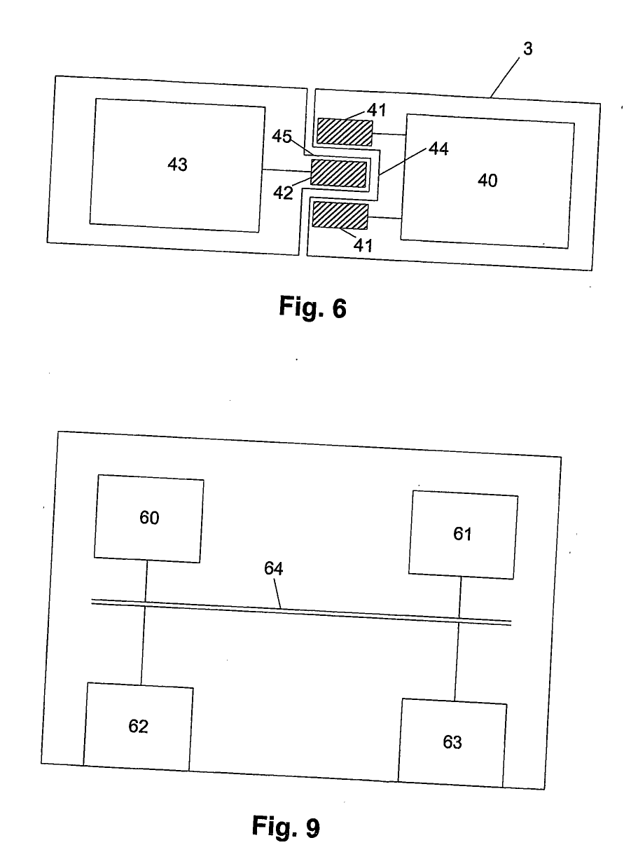

A feature that may be utilised is for the module 3 to be provided with an integral, rechargeable power supply, such as a battery. In this example, the battery can be coupled to a charging system through a wireless inductive coupling mechanism, as shown for example in FIG. 6. In this example, the battery 40 is coupled to a coil 41, which cooperates with a coil 42 provided in a charging system 43. By providing the coil 41 around a recess 44 in the module 3, which cooperates with a protrusion 45 in the charging system 43, this ensures maximum inductive coupling between the coils 41, 42, thereby improving charging efficiency. In this instance, the protrusion may advantageously form part of a hanging mechanism, allowing the battery to be charged when the blanket is hung up, and is not in use.

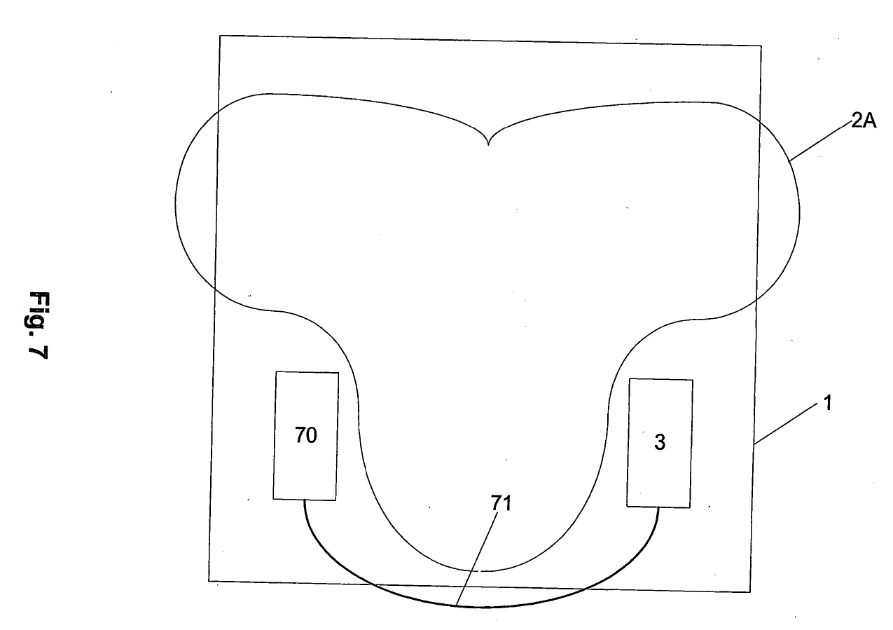

A separate power supply module 70 may also be provided depending on the implementation. If multiple modules 3, 11 are used, the modules 3, 70 may be positioned on either side of the saddle 2A, as shown in the plan view in FIG. 7. In this case, the modules can be connected via a cable 71 running around the rear of the saddle, as shown, to thereby avoid pressure points on the horse. This has the additional benefit of balancing the weight distribution of the blanket, thereby ensuring the blanket does not slip off when placed on the horse.

In the examples set out above, the module 3, and the optional power supply and GPS modules are preferably located in close proximity to the saddle, such that the modules are held in place by the saddle, reducing movement, although this is not essential. In general, continuous monitoring of horse position is not required, as this can unduly drain the batteries. This can therefore be overcome by entering weigh points and triggers in the GPS micro controller, that could activate the recording or reporting of velocity and heart rate under different circumstances. Weigh points could also be used to notify the trainer if a horse was not complying with the specified training regime. They can also be used to indicate if a horse is leaving or returning to the stable so that logged data can be downloaded out of the saddle blanket.

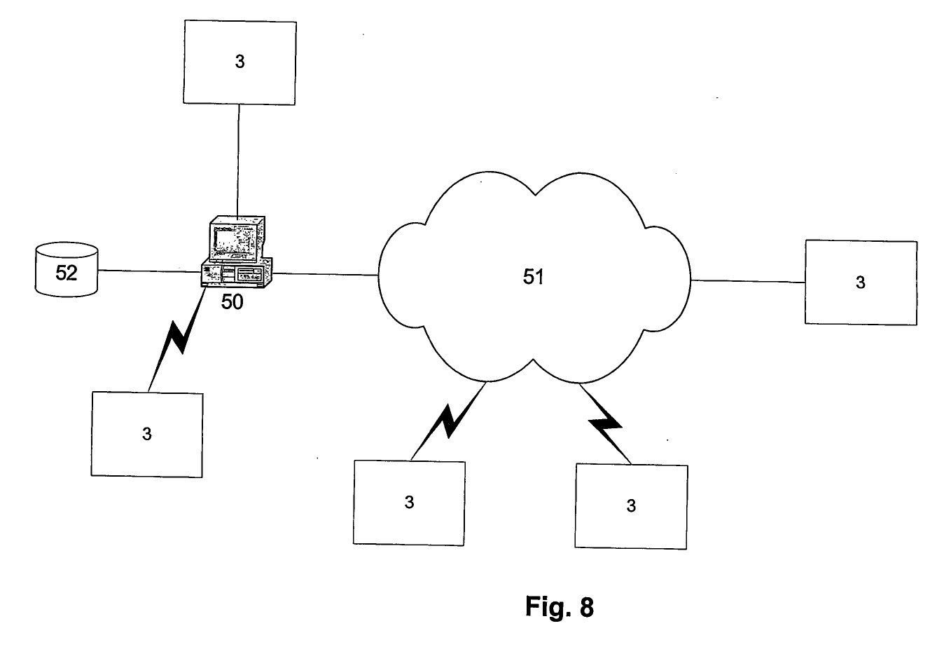

It will be appreciated from the above, that a number of different system may be monitored by a processing system 50, as shown for example, in FIG. 8. In this example, the processing system 50 communicates with a number of modules 3, which may be achieved via directly, or via a suitable communications network 51, such as a LAN, WAN, the Internet, the mobile phone network, or the like. The transfer of data is preferably performed wirelessly, but may also be performed through wired connections at the end of a ride as described above.

An example of a suitable processing system 50 is shown in FIG. 9. In particular, the processing system 60 generally includes at least a processor 60, a memory 61, an input/output (I/O) device 62, such as a keyboard and display, and an external interface 63 coupled together via a bus 64. The processing system can be coupled a database 52, and the network 51, via the external interface 65.

Accordingly, it will be appreciated that the processing system 10 may be formed from any suitable processing system, such as a suitably programmed PC, Internet terminal, lap-top, hand-held PC, or the like, which is typically operating applications software to enable data transfer and analysis.

In this case, each module 3 will be provided with a respective identifier, which is used to identify the source of the respective heart rate and position data, when it is transferred to the processing system 50. The identifier can be associated with the respective horse at the start of the ride by providing an indication of the module 3 being used, and an indication of the horse's identity. The system may use ID chips inserted under the animal skin for an identification process.

The processing system 50, can then store received data in a database 52, and use the data to determine the health status of a respective horse, which can then be displayed using the I/O device 62, or transferred to a remote processing system for subsequent display. For example, the processing system 50 can be adapted to generate an alert if the determined health status falls outside a predetermined range which represents a situation in which the horse may be harmed if riding continues.

This allows a single operator to monitor a number of horses being trained, to ensure that the horses are not trained in a manner which is detrimental to the horses health, and to generally observe horse fitness.

It will therefore be appreciated that this allows owners and trainers to gain access to results and details of monitoring via the Internet. Furthermore, it will be appreciated that the processing system 50 may be operated by an entity which provides analysis services, and which therefore operates to receive and analyse data, providing details of the determined results to owners and trainers.

Throughout the description and claims, the term horses should be understood to include racehorses, camels, llamas, greyhounds, performance animals, such as racing animals, and other non-performance animals, such as non-racing horses.

Furthermore, the description has focussed on the use of a heart rate monitor for measuring a vital sign of the horse. However, other monitoring techniques can be used to monitor different vital signs as an alternative, or an addition to measuring heart rate. In particular, monitoring can be performed by measuring:

blood pressure;

temperature;

breathing rate;

blood flow rate; and,

blood gas levels.

In addition to this, it is also possible to provide sensors for measuring environmental conditions, such as external temperature and other weather conditions. This allows a detailed record of the conditions under which the horse was trained to be ascertained and stored automatically.

### SPECIFIC EXAMPLE

In one example, recordings of heart rate and velocity during trotting on a sand track and gallops on a grass track were performed three times during a four week period in 8 Thoroughbred racehorses (3 geldings, 3 fillies and 2 colts; aged 2 to 4 years old).

Heart rates were recorded by Polar heart rate meters provided in a saddle blanket, with speed being measured using the GPS system, which can be used to calculate velocity with a speed accuracy: 0.36 kph. In this example, the blanket incorporates a 12 channel receiver interface to connect with a personal computer.

After inspection of the records of five-second averages of heart rate and velocity (0.2 Hz recordings) obtained during exercise, regressions of heart rate on velocity were constructed to derive V200.

The typical training exercise included 5 minutes trotting (mean trot speeds ranged from 4.1 to 4.6 m/s). After a brief period of walking, horses then galloped over 800-1000 metres on a grass track. This allowed for steady natural changes in gait, and represent the fitness and performance of the horse in general riding conditions.

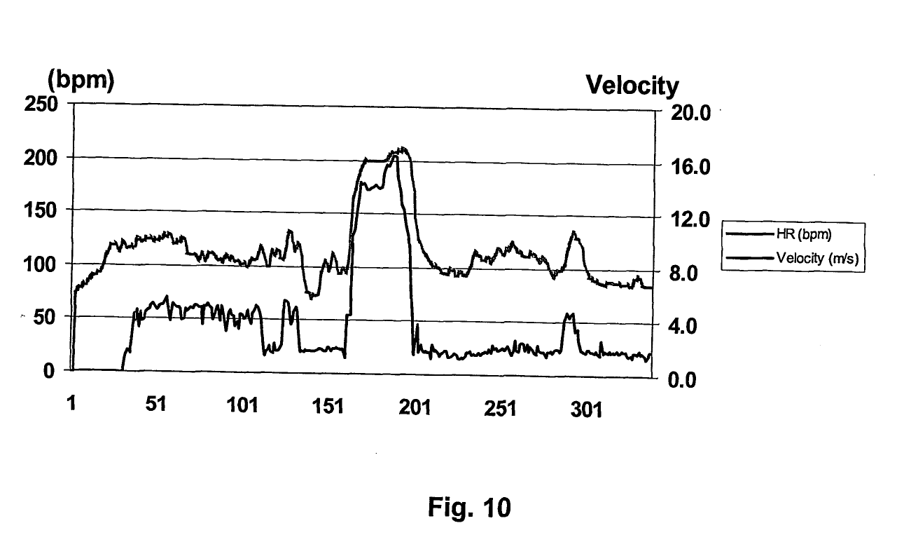

An example of the heart rate and velocity determined using the system described above is shown in FIG. 10.

To demonstrate the varying degrees of accuracy of the methodology over different conditions, four methods of calculation of V200 were used, as follows:
“Gallop with outliers” used only gallop heart rates, and included all heart rate and velocity records;
“Gallop without outliers” used only gallop heart rates and excluded the heart rate outliers (outliers were defined as heart rates that were more than 10 bpm higher than another heart rate at a similar speed);
“Trot plus Gallop with outliers” combined “Gallop with outliers” and trot data;
“Trot plus gallop without outliers” combined “Gallop without outliers” and trot data.

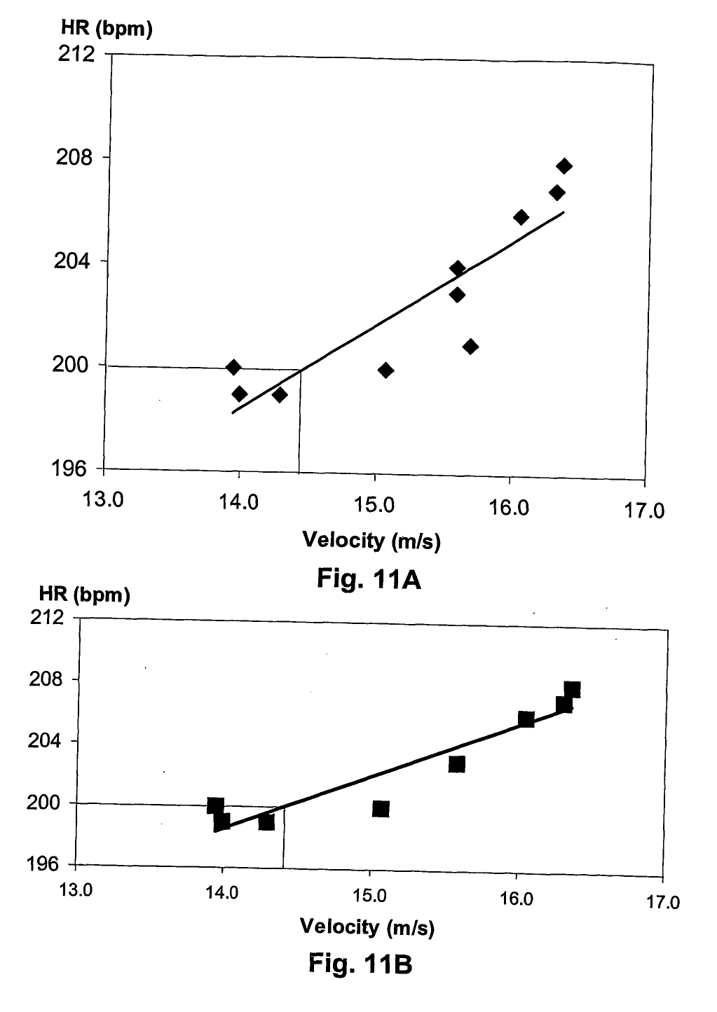

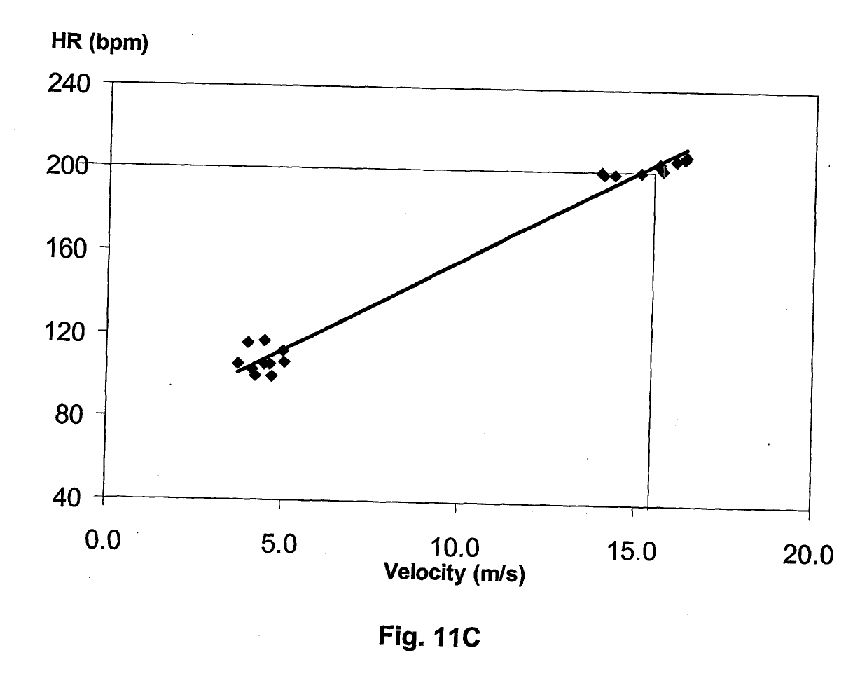

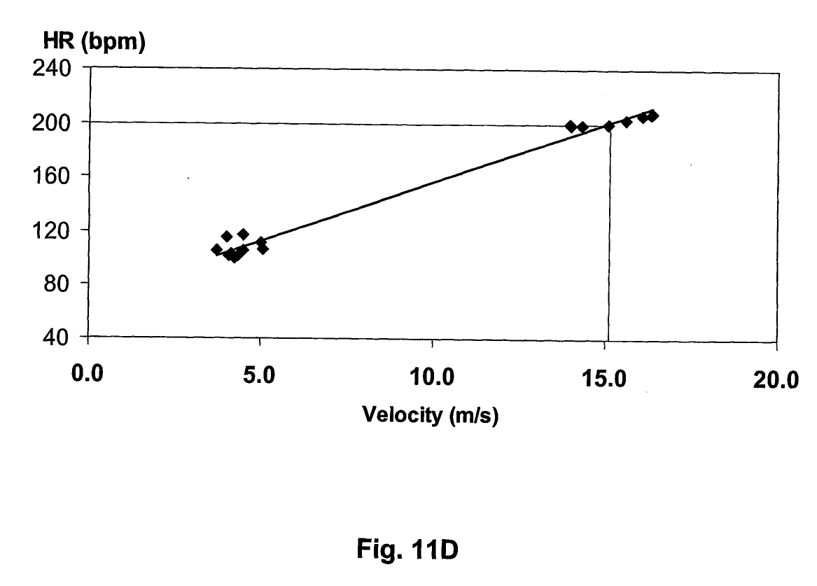

Graphs for each of these scenarios are set out in FIGS. 11A to 11D respectively.

For trot data, the average of the heart rate and velocity during the final 50 seconds of trotting were used, to thereby ensure gait changes did not have an undue impact.

A minimum speed of 11.1 m/s was used as the criteria for identification of the beginning of the gallop exercise, and only heart rates during periods of increasing speed were used during gallops, with occasional losses of heart rate or speed during exercise being excluded from the analyses, thereby reduce the effect of obstacles and the like.

From this, a linear regression is determined, as described above, allowing V200 and the regression coefficient R² to be determined. The results from this analysis are as follows:
Gallop with outliers -R²=0.79;
-V200*=14.4 m/s;
Gallop without outliers -R²=0.88;
V200*=14.3 m/s;
Trot plus Gallop with outliers -R²=0.98;
V200*=15.0 m/s;
Trot plus gallop without outliers -R²=0.99;
V200*=14.8 m/s.

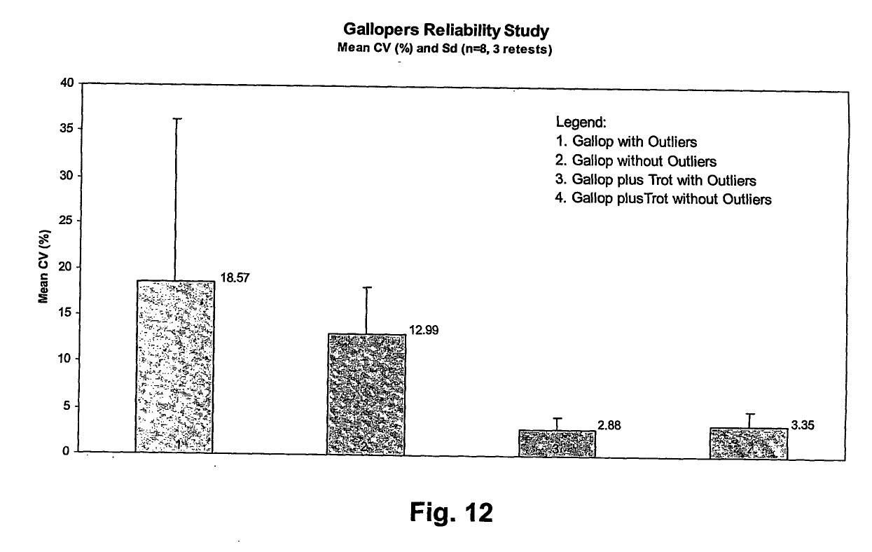

Variability of V200 within horse and also average for 8 horses was described by the coefficient of variation (CV), as shown in FIG. 12. In this case, for the four methods, the values were determined as follows:
Gallop with outliers -(2.8-51.9%), mean CV 23.1;
Gallop without outliers -(6.7-21.6%), mean CV 13.0;
Trot plus Gallop with outliers -(2.0-6.1%), mean CV 2.9; and
Trot plus gallop without outliers -(1.3-6.0%), mean CV 3.3.

Accordingly, this demonstrates the reliability of calculation of V200 in the field using the above described techniques and systems to allow V200 to be determined independent of a standardised test protocol.

This highlights that highly repeatable measurements of V200 are possible during field studies with GPS velocity and simultaneous heart rate recordings, as described, thereby allowing field fitness and health tests, whilst minimising disruptive fitness test in the field.

Furthermore, the use of GPS allows velocity to be measured every 5 seconds, with velocity to be measured over a constant distance, whilst also allowing acceleration and peak velocity to be measured.

Thus, the system and methodology described above provide a system which integrates monitoring electronics within a saddle blanket, which thereby eliminates some of the basic problems of equipment within a stable, such as:
Management and storage of electronics, as the monitoring equipment stays with saddle blanket and requires minimal maintenance;
Dirt, dust and rough usage, as the modules are typically provided in strong housings, such as aluminium boxes, which are also weather proof;
Simple heart rate monitor set-up; and,
Automated data collection and management.

Accordingly, in one example, the above described system can provide “on board” measurement of heart rate and velocity of freely moving horses during normal training exercises. This can be achieved using a small, lightweight data logger provided in the saddle blanket for recording velocity, using global positioning technology, and heart rate, using a heart rate monitor. This integration of the velocity sensing using GPS and heart rate sensing can be used to provide a health status.

The system can therefore provide an indicator of the performance of a horse during training, including for example, calculation of fitness indicators such as velocity at heart rate of 200 beats per minute (V-200), and velocity at maximal heart rate (VHR-max).

leading to early identification of lameness, disease, and poor physiological potential and consequently a reduction in wastage in the industry. This allows trainers and owners to implement best practice management for trainers including animal welfare and ethics and achieving better training outcomes.

Persons skilled in the art will appreciate that numerous variations and modifications will become apparent. All such variations and modifications which become apparent to persons skilled in the art, should be considered to fall within the spirit and scope that the invention broadly appearing before described.

The claims defining the invention are as follows:

1) Apparatus for monitoring the status of a horse, wherein the apparatus includes:
(a) a blanket having a first sensor, the first sensor being adapted to generate indicating data indicative of at least one health status indicator; and,
(b) a second sensor for generating position data indicative of the position of the horse, wherein, in use, a processing system is adapted to determine the health status of the horse in response to the indicating data and the position data.

2) Apparatus according to claim 1, wherein the health status indicator includes at least one of the horse's:
(a) heart rate;
(b) blood pressure;
(c) temperature; breathing rate;
(d) blood flow rate; and,
(e) blood oxygenation levels.

3) Apparatus according to claim 1, wherein the second sensor is formed from a GPS sensor.

4) Apparatus according to claim 1, wherein the second sensor is adapted to be worn by a rider in use, and wherein the blanket further includes a connector for coupling the second sensor to the blanket in use.

5) Apparatus according to claim 1, wherein the second sensor is provided in the blanket.

6) Apparatus according to claim 1, wherein the blanket further includes a power supply for coupling to the first and second sensors.

7) Apparatus according to claim 6, wherein the power supply includes at least one battery connected to a first part of an inductive coupling, and wherein, in use, the battery is recharged by connecting the first part of the inductive coupling to a second part of the inductive coupling, the second part being coupled to a power supply.

8) Apparatus according to claim 1, wherein the blanket further includes a communications device coupled to the first and second sensors to thereby transfer at least one of the indicating and position data to a remote computer system.

9) Apparatus according to claim 1, wherein the blanket further includes a store coupled to the first and second sensors to thereby store at least one of the indicating and position data to a remote computer system.

10) Apparatus according to claim 1, wherein the apparatus includes a processing system coupled to at least one of the first and second sensors for at least partially analysing at least one of the indicating and the position data.

11) Apparatus according to claim 10, wherein the processing system is coupled to a display, the display being adapted to provide an indication to the rider in accordance with at least one of the indicating and the position data.

12) Apparatus according to claim 1, wherein the first sensor is a heart rate sensor and wherein the blanket includes at least one electrode coupled to the heart rate sensor and positioned so as to be in contact with the horse in use.

13) Apparatus according to claim 12, wherein the blanket includes at least one wire embedded in the blanket material, the wire being adapted to connect the heart rate sensor to the at least one electrode.

14) Apparatus according to claim 13, wherein the blanket is a woven blanket and wherein the wire is integrated within the weave of the blanket.

15) Apparatus according to claim 1, wherein the first sensor is removably mounted to a pouch, the pouch including one or more connectors adapted to cooperate with corresponding detectors provided on the sensor, to thereby couple the sensor to the blanket.

16) Apparatus for monitoring the status of a horse, wherein the apparatus includes a processing system adapted to:
(a) receive, from a first sensor provided in a horse blanket, indicating data indicative of at least one health status indicator;
(b) receive, from a second sensor, position data indicative of the position of the horse; and,
(c) determine the health status of the horse in accordance with the indicating data and the position data.

17) Apparatus according to claim 16, wherein the processing system is adapted to receive the position and indicating data from apparatus according to claim 1.

18) Apparatus according to claim 16, wherein the processing system includes a communications device for receiving the indicating and position data.

19) Apparatus according to claim 16, wherein the processing system determines the health status of the horse using a predetermined algorithm, the predetermined algorithm defining a relationship between the at least one health status indicator and movement of the horse.

20) Apparatus according to claim 19, wherein the predetermined algorithm includes:
(a) determining at least a low heart rate during low speed exercise;
(b) determining a number of heart rates during high speed exercise;
(c) perform linear regression to calculate a linear regression line:
(d) calculate, using the linear regression line, the velocities at at least one of:
(i) heart rates of 200 beats per minute (V200); and,
(ii) HRmax (VHRmax); and,
(e) determine a fitness indicator in accordance with the calculated at least one velocity.

21) Apparatus according to claim 20, wherein the line regression line is determined in accordance with:
HR=a+bV,
where HR=heart rate;
a=constant;
b=constant; and,
V=velocity.

22) Apparatus according to claim 20, wherein the method further includes deleting any outlier values

23) Apparatus according to claim 22, wherein the method includes deleting all outlier values by at least one of:
(a) deleting all results with a velocity of less than 40 kph;
(b) deleting all results during the period after exercise (from the time of occurrence of HRmax);
(c) deleting all data equal to at least one of:
(i) HRmax;
(ii) HRmax−1;
(iii) HRmax−2; and,
(iv) HRmax−3;
(d) deleting all data where there has been an increase in velocity, but that increase was not accompanied by an increase in HR;
(e) deleting any data points which have a HR that is more than 10 beats per minute above the regression line at that speed, and recalculate the regression line if such outliers are deleted.

24) Apparatus according to claim 16, wherein the processing system is adapted to obtain indicating data and position data relating to a number of horses, the processing system being adapted to determine the health status of each of the number of horses.

25) Apparatus according to claim 16, the apparatus being used with the apparatus of claim 1.

26) Apparatus for monitoring the status of a horse, wherein the apparatus includes a processing system adapted to:
(a) receive, from a first sensor, indicating data indicative of the heart rate of the horse;
(b) receive, from a second sensor, position data indicative of the position of the horse;
(c) determine from the position data, movement data indicative of the rate of movement of the horse; and,
(d) determine the health status of the horse in accordance with a predetermined algorithm, the predetermined algorithm defining a relationship between the heart rate and the rate of movement of the horse.

27) Apparatus according to claim 26, wherein the predetermined algorithm includes:
(a) determining at least a low heart rate during low speed exercise;
(b) determining a number of heart rates during high speed exercise;
(c) perform linear regression to calculate a linear regression line:
(d) calculate, using the linear regression line, the velocities at at least one of:
(i) heart rates of 200 beats per minute (V200); and,
(ii) HRmax (VHRmax); and,
(e) determine a fitness indicator in accordance with the calculated at least one velocity.

28) Apparatus according to claim 27, the low heart rate being determined during trotting.

29) Apparatus according to claim 28, wherein the low heart rate is determined after the horse has been trotting for at least three minutes.

30) A system for monitoring the status of a horse, wherein the system includes:
(a) a blanket having a first sensor, the first sensor being adapted to generate indicating data indicative of at least one health status indicator; and,
(b) a second sensor for generating position data indicative of the position of the horse; and,
(c) a processing system, the processing system being responsive to the indicating and position data to thereby determine the heath status of the horse.

31) A system according to claim 30, the system including apparatus according to any one of the claims 1 to 29.

32) A method of monitoring the health status of a horse, wherein the method includes:
(a) using a blanket having a first sensor to generate indicating data indicative of at least one health status indicator; and,
(b) using a second sensor to generate position data indicative of the position of the horse; and,
(c) determining the health status of the horse in response to the indicating data and the position data.

33) A method according to claim 32, wherein the method is performed using the apparatus of any one of the claims 1 to 29.

34) A method of monitoring the health status of a horse, wherein the method includes, in a horse blanket:
(a) generating indicating data using a first sensor, the indicating data being indicative of at least one health status indicator;
(b) obtaining position data from a second sensor, the position data being indicative of the position of the horse; and,
(c) providing the indicating data and the position data to a processing system, the processing system being responsive to the indicating data and the position data to determine the health status of the horse.

35) A method according to claim 34, wherein the method is performed using the apparatus of any one of the claims 1 to 29.

36) A method of monitoring the health status of a horse, wherein the method includes, in a processing system:
(a) receiving, from a first sensor provided in a horse blanket, indicating data indicative of at least one health status indicator;
(b) receiving, from a second sensor, position data indicative of the position of the horse; and,
(c) determining the health status of the horse in accordance with the indicating data and the position data.

37) A method according to claim 36, wherein the method is performed using the apparatus of any one of the claims 1 to 29.

38) Apparatus for monitoring the status of a horse, wherein the apparatus includes a processing system adapted to:
(a) receive, from a first sensor, indicating data indicative of the heart rate of the horse;
(b) receive, from a second sensor, position data indicative of the position of the horse;
(c) determine from the position data, movement data indicative of the rate of movement of the horse; and,
(d) determine the health status of the horse in accordance with a predetermined algorithm, the predetermined algorithm defining a relationship between the heart rate and the rate of movement of the horse.

39) A method according to claim 38, wherein the predetermined algorithm includes:
(a) determining at least a low heart rate during low speed exercise;
(b) determining a number of heart rates during high speed exercise;
(c) perform linear regression to calculate a linear regression line:
(d) calculate, using the linear regression line, the velocities at at least one of:
(i) heart rates of 200 beats per minute (V200); and,
(ii) HRmax (VHRmax); and,
(e) determine a fitness indicator in accordance with the calculated at least one velocity.
